(19) United States
(12) Patent Application Publication
(10) Pub. No.: US 2016/0130208 A1
(43) Pub. Date: May 12, 2016

(54) SYNTHESIS OF ALPHA,BETA-UNSATURATED CARBOXYLIC ACID (METH)ACRYLATES FROM OLEFINS AND CO2

(71) Applicant: Evonik Degussa GmbH, Essen (DE)

(72) Inventors: Benjamin Schäffner, Dorsten (DE); Matthias Blug, Burscheid (DE); Dieter Vogt, Edinburgh (GB); Coen Hendriksen, Eindhoven (NL); Evgeny Pidko, Eindhoven (NL)

(73) Assignee: Evonik Degussa GMBH, Essen (DE)

(21) Appl. No.: 14/890,298

(22) PCT Filed: May 12, 2014
(86) PCT No.: PCT/EP2014/059596

§ 371 Date: Nov. 10, 2015

(30) Foreign Application Priority Data
Jun. 11, 2013 (DE) 10 2013 210 840.0

(51) Int. Cl. C07C 51/15

(52) U.S. Cl. CPC C07C 51/15

(57) ABSTRACT

The invention relates to a method for producing α,β-unsaturated carboxylic acids or salts thereof, comprising a step in which a metallalactone is reacted in a solvent in the presence of a halide; to a composition that comprises α,β-unsaturated carboxylic acids or salts thereof and halide ions; and to the use of said composition for the production of superabsorbent materials or as a monomer composition for producing polymers.

The present invention relates to a process for preparing α,β-unsaturated carboxylic acids (e.g. acrylic or methacrylic acid) or a salt of the α,β-unsaturated carboxylic acids, which has a process step in which a complex is reacted in a solvent in the presence of a halide, a composition comprising α,β-unsaturated carboxylic acids or a salt thereof and also halide ions and the use of these compositions for producing superabsorbent materials or as monomer composition for preparing polymers, e.g. polymethyl methacrylate.

To reduce gases which damage the climate, e.g. carbon dioxide, numerous processes in which CO₂ is used as raw material for producing wanted chemical products have been developed recently. One process is, for example, the preparation of acrylates by reaction of carbon dioxide with olefins in the presence of a nickel-bisphosphine catalyst and a base, as described by Michael L. Lejkowski et al., “The First Catalytic Synthesis of an Acrylate from CO₂ and an Alkene—A Rational Approach”, Chem. Eur. J. 2012; Wiley-VCH Verlag GmbH&Co. KGaA, Weinheim; Wiley Online Library; DOI: 10.1002/chem.201201757. The catalysis cycle presented comprises the steps of reaction of an olefin complex with CO₂ to form the lactone complex, conversion of the lactone complex into the acrylate complex and subsequent replacement of the acrylate ligand with the olefin ligand to give the olefin complex. Strong bases such as sodium butoxide or NaOH are used in the conversion of the lactone complex into the acrylate complex. A disadvantage of the use of these strong bases is that in a CO₂ atmosphere they tend to react with the carbon dioxide to form carbonates and are thus no longer available for the catalytic reaction. Relatively complicated engineering measures in which the catalysis cycle is divided into a CO₂-rich part and a low-CO₂ part have to be undertaken in order to avoid this secondary reaction. In the CO₂-rich part, the reaction of the olefin complex with CO₂ to form the lactone complex is carried out. In the low-CO₂ part, the conversion of the lactone complex into the acrylate complex and subsequently the replacement of the acrylate ligand with the olefin ligand are carried out. This sequential approach leads, apart from the high outlay to significant slurry of the reaction since the catalytic process is achieved only stepwise.

As early as in WO 2011/107559, Limbach et al., who was also involved as author in the abovementioned publication, described processes for preparing an alkali metal salt or alkaline earth metal salt of an α,β-ethylenically unsaturated carboxylic acid, in which a) an alkene, carbon dioxide and carboxylation catalyst are reacted to form an alkene/carbon dioxide/carboxylation catalyst adduct, b) the adduct is decomposed by means of an auxiliary base with liberation of the carboxylation catalyst to form the auxiliary base salt of the α,β-ethylenically unsaturated carboxylic acid, c) the auxiliary base salt of the α,β-ethylenically unsaturated carboxylic acid is reacted with an alkali metal base or alkaline earth metal base to liberate the auxiliary base and form the alkali metal salt or alkaline earth metal salt of the α,β-ethylenically unsaturated carboxylic acid.

As auxiliary bases, WO 2011/107559 mentioned, for example, anion bases such as salts with inorganic or organic ammonium ions or alkali metals or alkaline earth metals or neutral bases, where inorganic anion bases can be, inter alia, carbonates, phosphates, nitrates or halides and organic anion bases can be, inter alia, phenoxides, carboxylates, sulphates of organic molecular moieties, sulphonates, phosphates, phosphonates and organic neutral bases can be, inter alia, primary, secondary or tertiary amines, also ethers, esters, imines, amides, carbonyl compounds, carboxylates or carbon monoxide. Preference is given to using a primary, secondary or tertiary amine, particularly preferably a tertiary amine, as auxiliary base.

A disadvantage of the use of amines as auxiliary bases is that the auxiliary bases have to be removed again in one or more steps. This preferably occurs using alkali metal carbonates, alkali metal hydroxides or oxides, preferably sodium hydroxide.

A further publication by Bernskoetter et al. “Lewis Acid Induced β-Elimination from a Nickelalactone: Efforts toward Acrylate Production from CO₂ and Ethylene” (Organometallics, 2013, DOI: 10.1021/om400025 h) describes the synthesis of acrylates using strong (Lewis) acids such as tris(pentafluorophenyl)borane. This compound was used in the study group in order to achieve additional activation of the nickelalactone. This enables the ring to be opened and the acrylate finally to be bound to the complex. A disadvantage is the use of tris(pentafluorophenyl)borane. The route to these compounds is complicated and not ensured on a large industrial scale. In addition, the reaction was carried out stepwise and the nickelalactone complex required for the reaction was synthesized separately beforehand. The complete catalysis cycle was thus not carried out.

It was an object of the present invention to provide a process for preparing α,β-unsaturated carboxylic acids such as (meth)acrylates from olefins and CO₂, which process avoids one or more disadvantages of the existing processes of the prior art.

It has surprisingly been found that the addition of salts from the group of alkali metal halides to the reaction mixture likewise enables the above-described reaction to be catalyzed. It was found here that simple halides, in particular iodides such as lithium iodide, sodium iodide or also potassium iodide, can function as Lewis acid in order to destabilize or cleave the nickelalactone formed.

An advantage over the method of Limbach et al. is, in particular, that all iodides used have only a low basicity and no binding of CO₂ to form carbonates, as is the case for strong bases (e.g. sodium butoxide or lithium hexamethyldisilazane (LiHMDS)), thus occurs. Compared to the above-described Lewis acid tris(pentafluorophenyl)borane, the halides used are more readily available and simpler to handle even when large amounts are used. Since the compounds are also stable over a relatively wide temperature range, the salts can be separated off and reused more easily.

In addition, simple ammonium iodides such as tetrabutylammonium iodide are also suitable for the reaction. Last but not least, the use of sodium iodide as salt and Lewis acid can lead directly to formation of sodium acrylate, and intermediate for the synthesis of superabsorbents.

The process of the invention has, in particular, the advantage that the synthesis of α,β-unsaturated carboxylic acids or salts thereof can be carried out directly from the starting materials olefin and CO₂ without isolation of specific intermediates.

The process of the invention, the composition of the invention and the use thereof are described by way of example below without the invention being intended to be restricted to these illustrated embodiments. Where ranges, general formulae or classes of compounds are indicated below, these are intended to encompass not only the corresponding ranges or groups of compounds which are explicitly mentioned but also all subranges and subgroups of compounds which can be obtained by leaving out individual values (ranges) or compounds. Where documents are cited in the present description, the contents thereof, in particular with regard to the subjects referred to, are fully incorporated by reference into the disclosure content of the present invention. Where percentages are indicated in the following, these are, unless indicated otherwise, % by weight. Where averages are indicated below, these are, unless indicated otherwise, the number average. Where material properties such as viscosities or the like are indicated below, these are, unless indicated otherwise, the material properties at 25° C. Where chemical (empirical) formulae are used in the context of the present invention, the indices indicated can be both absolute numbers and averages. In the case of polymeric compounds, the indices are preferably averages. If the term “(meth)acrylic acid” or (meth)acrylate is used in the present patent application, this encompasses both methacrylic acid and acrylic acid or both methacrylate and acrylate.

The process of the invention for preparing α,β-unsaturated carboxylic acids or a salt of the α,β-unsaturated carboxylic acids, preferably (meth)acrylic acid or a salt of (meth)acrylic acid, preferably acrylic acid or a salt of acrylic acid, is characterized in that it has a process step in which a substance of the formula (I)

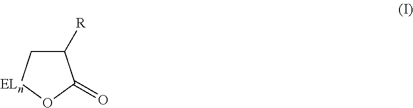

where
E=element of group 4, 6, 7, 8, 9 or 10 of the Periodic Table of the Elements, preferably nickel,
L=nitrogen or phosphorus-containing, preferably bidentate phosphorus ligand,
n=1 to 4, preferably 1,
R=H or an aryl or alkyl radical, preferably H or a branched or unbranched alkyl radical having from 1 to 10 carbon atoms, particularly preferably H or a methyl group and very particularly preferably H,
is, preferably as an intermediate (or temporarily), reacted in a solvent in the presence of a halide, preferably selected from the group consisting of alkali metal halides, alkaline earth metal halides and ammonium halides. Preference is given to using NaI, LiC1, LiI and (nBu)₄NI as halides. Particular preference is given to using iodides, in particular NaI and/or LiI, as halides.

L in the process of the invention is preferably a ligand selected from among phosphanes and phosphonates, preferably bisphosphanes and bisphosphonates, preferably selected from among trialkylbisphosphane, dialkylarylbisphosphane, alkyldiarylbisphosphane and triarylbisphosphane ligands. L is very particularly preferable selected from among bis(dicyclohexylphosphino)ethane (dcpe), bis(di-tert-butylphosphino)ethane and bis(diphenylphosphino)ethane.

As solvent, it is possible to use all known solvents, the solvent is preferably selected from among halogenated hydrocarbons, halogenated aromatics and cyclic ethers, preferably chlorobenzene or dichloromethane or tetrahydrofuran. Particular preference is given to using chloroform, dichloromethane or chlorobenzene, preferably chlorobenzene, as solvent.

In the process of the invention, the process step is very particularly preferably carried out by reacting a substance of the formula (I) in which E=nickel)(Ni⁰, n=1, L=bis(diphenylphosphino)ethane or bis(dicyclohexylphosphino)ethane in a solvent, preferably chlorobenzene, dichloromethane or tetrahydrofuran, preferably chlorobenzene, in the presence of an iodide selected from among NaI, LiI and (nBu)₄NI.

The reaction can be carried out at atmospheric pressure or superatmospheric pressure. The reaction of the compound of the formula (I) (the nickelalactone) is preferably carried out at partial pressures of from 1 to 50 bar of CO₂ and from 1 to 50 bar of the respective olefins.

The reaction can be carried out at any desired temperature. The reaction is preferably carried out at a temperature of from 0 to 150° C., preferably from 15 to 100° C. and particularly preferably at a temperature of from 25 to 60° C.

The molar ratio of halide ions to element E is preferably from 0.1:1 to 50:1, more preferably from 1:1 to 20:1.

It can be advantageous for the substance of the formula (I) to be obtained by reaction of a complex of the formula (II)

ELₙ (II)

where E, L and n are as defined above, with an olefin and carbon dioxide. As olefins, preference is given to using hydrocarbons which have at least one unsaturated carbon-carbon bond. Hydrocarbons which have from 1 to 10 carbon atoms are preferably used as olefins. Particular preference is given to using ethene or propene as olefins, with very particular preference being given to ethene.

This reaction can, for example, be carried out as described in Michael L. Lejkowski et al., “The First Catalytic Synthesis of an Acrylate from CO₂ and an Alkene—A Rational Approach”, Chem. Eur. J. 2012; Wiley-VCH Verlag GmbH&Co. KGaA, Weinheim; Wiley Online Library; DOI: 10.1002/chem.201201757. In addition, a simple method of preparing the compound of the formula (I) may be found in an article by Heinz Hoberg und Dietmar Schäfer “Nickel(0)-induzierte C-C-Verknüpfung zwischen Kohlendioxid und Ethylen sowie Mono-oder Di-substituierte Alkenen” J. Organomet. Chem. 1983, 251, C51-053 (Elsevier Sequoia).

For the preparation of the compound of the formula (I), preference is given to using (1,5-cyclooctadiene)₂nickel (Ni(cod)₂) and dissolving this together with ligand L, preferably 1,2-bis(dicyclohexylphosphino)ethane, bis(di-tert-butylphosphino)ethane or bis(diphenylphosphino)ethane, in tetrahydrofuran. Olefin, preferably ethene or propene, more preferably ethene, and CO₂ are subsequently added. The molar ratio of the compounds relative to one another is complex of the formula (II): CO₂: olefin, preferably ethene, =1:1:5. After removal of tetrahydrofuran, the compound of the formula (I) can be obtained as yellow-green compound in a yield of 50%.

The substance of the formula (I) is preferably prepared by direct reaction of a catalyst precursor of the element E with a preferably bidentate phosphane or phosphonate ligand in a halogenated solvent or cyclic ether in a CO₂/olefin atmosphere, preferably a CO₂/ethene or CO₂/propene atmosphere, more preferably in a CO₂/ethene atmosphere.

Particular preference is given to reacting a complex of the formula (II) in which E=nickel)(Ni⁰, n=2 and L=bis(diphenylphosphino)ethane or bis(dicyclohexylphosphino)ethane, preferably bis(dicyclohexylphosphino)ethane, with an olefin, preferably ethene or propene, more preferably ethene, and carbon dioxide in a molar ratio of 10/40 at from 1 to 75 bar, preferably from 30 to 60 bar, more preferably about 50 bar, in chlorobenzene, dichloromethane or tetrahydrofuran, preferably chlorobenzene, at from 30 to 60° C.

The process of the invention makes it possible to obtain the compositions of the invention, which comprise halide ions and α,β-unsaturated carboxylic acid, preferably (meth)acrylic acid, particularly preferably acrylic acid or salts thereof (with alkali metal ions, alkaline earth metal ions or ammonium ions), in particular the compositions according to the invention described below.

The compositions of the invention containing the α,β-unsaturated carboxylic acid or a salt thereof are characterized in that they comprise halide ions, preferably iodide ions. The proportion of halide ions, preferably iodide ions, is preferably from 20 to 1000 mol %, more preferably from 50 to 500 mol %, based on the substance of the formula (I).

In the case of compositions according to the invention which have been obtained by a process according to the invention in which the substance of the formula (I) is not isolated, the compositions of the invention also comprise α,β-unsaturated carboxylic acid or a salt thereof and a halide ion, preferably iodide ions. The proportion of halide ions, preferably iodide ions, is preferably from 50 to 5000 mol %, more preferably from 100 mol % to 500 mol %, based on E. In this process, preference is given to using ligands L which are selected from the group consisting of bis(dicyclohexylphosphino)ethane and bis(di-tert-butylphosphino)ethane (d^{t}Bupe). The content of E can be determined by known suitable analytical methods. When E is, for example, nickel, the nickel content can, for example, be determined by means of atomic absorption spectrometry (AAS) at 232.0 nm, e.g. as described in Welz, B.; Sperling, M., Atomabsorptionsspektrometrie, Wiley-VHC: Weinheim, (1997); p. 565. The halide determination can be carried out by known suitable analytical methods. The determination of chloride, iodide and bromide is preferably carried out by the “Volhard titration” method, as is described in known chemistry textbooks.

The compositions of the invention, in particular those containing acrylic acid or salts thereof, can, for example, be used for producing superabsorbent materials or as monomer compositions for preparing polymers. The preparation of such polymers or superabsorbent materials is described, for example, in the book “Modern Superabsorbent Polymer Technology”, John Wiley & Sons; edition: 1st edition (11 Dec. 1997), ISBN-13: 978-0471194118.

The compositions of the invention, in particular those containing methacrylic acid or salts thereof, can be used, for example, for preparing polymethyl methacrylates and semifinished parts or plates produced therefrom.

The present invention is described by way of example in the following examples without the invention, whose scope is indicated by the total description and the claims, being restricted to the embodiments mentioned in the examples.

### EXAMPLES

### Measurement Methods

¹H-, ¹³C- and ³¹P-NMR spectra were recorded at room temperature by means of an NMR spectrometer (Avance III, 400 MHz, from Bruker). All spectra were referenced by means of TMS or by means of the solvent peak of the deuterated solvent. Infrared measurements were recorded using an IR spectrometer (2000 FT-IR, from Perkin-Elmer). Acrylic acid was detected by means of GC using a gas chromatograph Shimadzu GC-2010 (from Shimadzu) and a CP-Wax (ffap) cb column (from Agilent).

### Example 1

### Preparation of a Substance of the Formula (I) for Decomposition Experiments

In a glass flask, 300 mg (1.21 mmol) of tetramethylethylenediaminenickelalactone were dissolved in 15 ml of tetrahydrofuran and admixed with 488.8 mg (1.23 mmol) of diphenyldiphosphinoethane under an inert gas atmosphere. The yellow suspension was subsequently stirred at 22° C. for 16 hours. The solvent was subsequently removed under reduced pressure and the remaining yellow solid was washed 5 times with 10 ml of tetrahydrofuran. The product was subsequently dried under reduced pressure.

### Example 2

### In Situ Generation of a Substance Having the Formula (I) for Catalytic Experiments with Simultaneous Conversion into Acrylic Acid

In a 4 ml reaction vessel provided with a magnetic stirrer, 13.5 mg (0.049 mmol) of (1,5-cyclooctadiene)₂nickel (Ni(cod)₂), 0.049 mmol of the respective ligand (1 equivalent) and 0.25 mmol (5 equivalents) of halide salt were admixed with 2 ml of solvent and the mixture was subsequently placed in an autoclave. The pressure in the autoclave was set to 10 bar of ethylene. After one hour, the pressure was set to 50 bar of CO₂ and the system was heated to 50° C. After 96 hours, the pressure was released and the mixture of substances was analyzed. For this purpose, the reaction was stopped by addition of 0.02 ml of trifluoroacetic acid and unreacted nickelalactone was converted into free propionic acid. The reaction mixture was dissolved in 1.0 ml of tetrahydrofuran and admixed with 1 mg of acetic acid in 0.5 ml of tetrahydrofuran (internal standard). The mixture was subsequently filtered through silica gel and analyzed by means of GC and NMR.

### Example 3

### Reaction of a Substance of the Formula (I) in a Solvent in the Presence of a Halide

In a reaction vessel having a total volume of 2 ml, 5.3 mg (0.125 mmol) of lithium chloride were added to a solution consisting of 1 ml of dichloromethane (DCM) and 13.3 mg (0.025 mmol) of (diphenylphosphinoethane)Ni(C₃H₄O₂) (I) under an inert gas atmosphere. The mixture was subsequently stirred at 22° C. for 20 hours. The mixture of substances was subsequently worked up and analyzed as per Example 2.

Further studies on the decomposition reaction were carried out in a manner analogous to Example 3 with variation of salt and solvent at 50° C., as per Table 1. The results for the decomposition reaction of the substance of the formula (I) in which E=nickel)(Ni⁰, n=1, L=bis(diphenylphosphino)ethane when using different salts and solvents are shown in Table 1.

TABLE 1
Variation of the solvent and of the
salts at 50° C. as per Example 3.

| Complex | Salt | Solvent | T (° C.) | Acrylate (%)^{a} |
|---|---|---|---|---|
| (I) | LiCl | THF | 50 | 17 |
| | | DMF | 50 | 13 |
| (I) | LiBr | THF | 50 | 4 |
| (I) | LiI | THF | 50 | 16 |
| | | MeOH | 50 | 7 |
| | | DMF | 50 | 32 |
| | | CHCl₃ | 50 | 74 |
| | | Propylene carbonate | 50 | 4 |
| | | Toluene | 50 | 47 |
| (I) | LiOTf | DMF | 50 | 18 |
| (I) | NaI | THF | 50 | 19 |
| | | DMF | 50 | 30 |
| (I) | NaCl | THF | 50 | 5 |
| (I) | NaBr | THF | 50 | 6 |
| (I) | KCl | THF | 50 | 5 |
| (I) | KBr | THF | 50 | 5 |
| (I) | KI | THF | 50 | 16 |
| | | DMF | 50 | 31 |
| (I) | NaOTf | THF | 50 | 12 |
| | | DMF | 50 | 39 |
| (I) | KOTf | THF | 50 | 9 |
| | | DMF | 50 | 32 |
| (I) | LiB(C₆F₅)₄ | | 50 | |

^{a}Relative GC peaks were determined by correlation between acrylate and propionate using acetic acid as internal standard.

Further studies on the decomposition reaction were carried out in a manner analogous to Example 3 with variation of salt and solvent at 22° C., as per Table 2. The results for the decomposition reaction of the substance of the formula (I) in which E=nickel)(Ni⁰, n=1, L=bis(diphenylphosphino)ethane when using various salts and solvents are shown in Table 2.

TABLE 2
Variation of the solvent and of the
salts at 22° C. as per Example 3.

| Complex | Salt | Solvent | T (° C.) | Acrylate (%)^{a} |
|---|---|---|---|---|
| (I) | LiCl | THF | 22 | 4 |
| | | DMF | 22 | 4 |
| (I) | LiBr | CHCl₃ | 22 | 40 |
| | | Toluene | 22 | 3 |
| (I) | LiI | THF | 22 | 7 |
| | | MeOH | 22 | 4 |
| | | DMF | 22 | 27 |
| | | MeCN | 22 | 5 |
| | | Toluene | 25 | 12 |
| | | Chlorobenzene | 22 | 3 |
| | | Acetone | 22 | 3 |
| | | Et₂O | 22 | 4 |
| | | CHCl₃ | 22 | 68 |
| | | Propylene carbonate | 22 | 2 |
| (I) | LiOTf | DMF | 22 | 24 |
| | | THF | 22 | 3 |
| (I) | | | | |
| (I) | LiPF₆ | DMF | 22 | 26 |
| (I) | NaI | THF | 22 | 7 |
| | | CHCl₃ | 22 | 2 |
| (I) | TBAI | DMF | 22 | 29 |

^{a}Relative GC peaks were determined by correlation between acrylate and propionate using acetic acid as internal standard.

Further studies on the decomposition reaction were carried out in a manner analogous to Example 3 with variation of salt and solvent at 100° C., as per Table 3. The results for the decomposition reaction of the substance of the formula (I) in which E=nickel)(Ni⁰, n=1, L=bis(diphenylphosphino)ethane when using different salts and solvents are shown in Table 3.

TABLE 3
Variation of the solvent and of the
salts at 100° C. as per Example 3.

| Complex | Salt | Solvent | T (° C.) | Acrylate (%)^{a} |
|---|---|---|---|---|
| (I) | LiI | Toluene | 100 | 56 |
| (I) | LiBr | Toluene | 100 | 35 |
| (I) | NaI | Toluene | 100 | 5 |

^{a}Relative GC peaks were determined by correlation between acrylate and propionate using acetic acid as internal standard.

In the experiments at elevated temperature, it was found that the best results can be achieved using halide-containing salts, in particular iodides, while only low yields of acrylates were obtained using the triflates (OTf) used for comparison. Here, only halide-free solvents such as alcohols or ethers such as tetrahydrofuran were used.

Further studies on the decomposition reaction were carried out in a manner analogous to Example 3 with variation of the salt at 22° C., as per Table 4. The results for the decomposition reaction of the substance of the formula (I) in which E=nickel (Ni0), n=1, L=bis(diphenylphosphino)ethane when using various salts and dichloromethane (DCM) are shown below.

TABLE 4
Variation of the salts in dichloromethane
at 22° C. as per Example 3.

| Starting material | Salt | Solvent | T (° C.) | Acrylate (%)^{a} |
|---|---|---|---|---|
| (I) | LiCl | DCM | 22 | 3 |
| (I) | LiI | DCM | 22 | 65 |
| (I) | NaI | DCM | 22 | 4 |
| (I) | NaOTf | DCM | 22 | 4 |
| (I) | TBAI | DCM | 22 | 68 |
| (I) | LiOTf | DCM | 22 | 2 |
| (I) | Li₂CO₃ | DCM | 22 | 6 |
| (I) | LiB(C₆F₅)₄ | DCM | 22 | 2 |

^{a}Relative GC peaks were determined by correlation between acrylate and propionate using acetic acid as internal standard.

In the experiments, the particular suitability of halides, in particular iodides, becomes clear. Thus, high acrylate yields could be achieved even at room temperature by the use of lithium iodide or tetrabutylammonium iodide (TBAI). In addition, halogenated solvents are preferred for the reaction.

Owing to the poor suitability of dichloromethane as solvent in large-scale industrial applications, experiments using chlorobenzene as solvent were carried out in situ.

Experiments on the in situ formation of acrylic acid from the starting materials were carried out in a manner analogous to Example 2, as per Table 5. The corresponding intermediate (I) was in this case not isolated but converted directly into the acrylate by addition of the halide.

TABLE 5
Variation of the salts in chlorobenzene at 50°
C. for the in situ experiments as per Example 2.

| Catalyst | Ligand | Salt | Acrylate (%)^{a} |
|---|---|---|---|
| Ni(cod)₂ | d^{t}Bupe | LiCl | 2 |
| Ni(cod)₂ | d^{t}Bupe | LiI | 6 |
| Ni(cod)₂ | dcpe | LiI | 80 |

^{a}Relative GC peaks were determined by correlation between acrylate and propionate using acetic acid as internal standard.

It can be seen from the results in Table 5 that direct reaction of ethylene and CO₂ via the intermediate (I) formed in situ is possible in the presence of lithium chloride and lithium iodide in halogenated solvents.

1. A process for preparing α,β-unsaturated carboxylic acids or salts thereof, wherein it has a process step in which a substance of the formula (I) 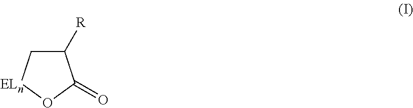
where
E=element of group 4, 6, 7, 8, 9 or 10 of the Periodic Table of the Elements,
L=nitrogen- or phosphorus-containing ligand,
n=1 to 4,
R=H or an aryl or alkyl radical,
is reacted in a solvent in the presence of a halide.

2. The process according to claim 1, wherein R in formula (I) is H or an alkyl radical having from 1 to 10 carbon atoms.

3. The process according to claim 1, wherein R in formula (I) is H or a methyl radical.

4. The process according to claim 1, wherein E in formula (I) is nickel.

5. The process according to claim 1, wherein L is a bidentate ligand.

6. The process according to claim 1, wherein L is a bisphosphane or bisphosphonate ligand, preferably selected from among trialkylbisphosphane, dialkylarylbisphosphane, alkyldiarylbisphosphane, triarylbisphosphane ligands.

7. The process according to claim 1, wherein L is a ligand selected from among bis(di-tert-butylphosphino)ethane, bis(dicyclohexylphosphino)ethane or bis(diphenylphosphino)ethane.

8. The process according to claim 1, wherein the halide is an iodide.

9. The process according to claim 1, wherein the halide is selected from among NaI, LiI and (nBu)₄NI.

10. The process according to claim 1, wherein the solvent is selected from among cyclic ethers, chlorinated aromatics and aliphatic chlorinated hydrocarbons, particularly preferably chloroform, chlorobenzene and dichloromethane.

11. The process according to claim 1, wherein the compound of the formula (I) is obtained by reaction of a complex of the formula (II)
ELₙ (II)
with an olefin and carbon dioxide.

12. The process according to claim 11, characterized in that the process is carried out without isolation of the compound of the formula (I).

13. The process according to claim 10, wherein propene or ethene, preferably ethene, is used as olefin.

14. The composition containing E, an α,β-unsaturated carboxylic acid or a salt thereof, wherein it comprises halide ions, with E as defined in claim 1.

15. The composition according to claim 14, wherein the α,β-unsaturated carboxylic acid or salt thereof is acrylic acid or methacrylic acid or a salt thereof.

16. The composition according to claim 14, wherein the proportion of iodide ions is from 20 to 1000 mol %, based on 100 mol % of E.

17. The composition according to claim 14, wherein the proportion of the α,β-unsaturated carboxylic acid or salt thereof is from 2 to 100% by weight based on the composition minus the amount of halide ions, solvent, E and L, with L.

18. The use of a composition according to claim 14 for producing superabsorbent materials or as monomer composition for preparing polymers.
